(19) United States
(12) Patent Application Publication
(10) Pub. No.: US 2017/0252269 A1
(43) Pub. Date: Sep. 7, 2017

(54) BIOACTIVE COATED GUTTA-PERCHA AS ROOT CANAL FILLING MATTER

(71) Applicant: UNIVERSITI MALAYA, Kuala Lumpur (MY)

(72) Inventors: Zeti Adura CHE AB AZIZ, Kuala Lumpur (MY); Muralithran Govindan KUTTY, Kuala Lumpur (MY); Noor Hayaty ABU KASIM, Kuala Lumpur (MY); Afaf Yahya MOHAMMED AL-HADDAD, Kuala Lumpur (MY)

(21) Appl. No.: 15/510,994

(22) PCT Filed: Sep. 25, 2015
(86) PCT No.: PCT/MY2015/000079

§ 371 Date: Mar. 13, 2017

(30) Foreign Application Priority Data
Sep. 12, 2014 (MY) PI 2014702610

(51) Int. Cl. A61K 6/00

(52) U.S. Cl. CPC A61K 6/0038; A61K 6/0002; A61K 6/0085; A61K 6/0047

(57) ABSTRACT

The present invention relates to the deposition of a micro-crystalline hydroxyapatite and tricalcium phosphate coating onto gutta-percha cones, to be used as dental composite for root canal filling. The method proposed for coating involves the surface pretreatment of gutta-percha cones with sodium hydroxide; immersion of gutta-percha cones in simulated body fluid, which contains calcium and phosphate ions; and replacing consumed simulated body fluid after an interval of time, at physiological pH and temperature. The nucleation process results in the biomimetic deposition of a thin and uniform layer of calcium phosphates and hydroxyapatite. Improved characteristics such as in sealing ability, bonding strength, and ability to form hermetic seal, therefore allows the dental composite produced, to be used appropriately as filling matter in root canal treatments.

### FIELD OF INVENTION

The present invention relates to gutta-percha coated with hydroxyapatite, to be used as root canal filling matter in the area of endodontics for the purpose of restorative dentistry.

### BACKGROUND OF INVENTION

Root canal treatment or endodontic therapy is a sequence of treatment for the infected or decayed pulp of a tooth which results in the elimination of infection and protection from reinvasion. During root canal treatment, the inflamed or infected pulp is removed and after the inside of the tooth is carefully cleaned and disinfected, the canal space is filled and sealed with a rubber-like material called gutta-percha or other similar material, together with adhesive cement called a sealer. The tooth is then restored with a crown or filling for support. The finished assembly of the natural root, post, core and crown forms a restored tooth that may continue to function like any other tooth, fabricated ideally in the same colour. Gutta-percha (GP) is the dried resin of the Taban tree (Order: Sapotaceae). It is a trans-isomer of polyisoprene with chemical structure of 1,4, trans-polyisoprene, and is the standard polymeric material used in composites for root canal obturation. Dental gutta-percha matrix contains actual gutta-percha, zinc oxide, heavy metal salts, and wax or resin. General properties of gutta-percha points include: biocompatible, dimensionally stable, capable of sealing the canal laterally and apically, insoluble, radiopaque, and easily removed from the canal if necessary. However, the disadvantages of gutta-percha include poor sealing and adhesion, resulting in void spaces and requirement for sealers or cements to fill in the spaces around the filling material. Hence, the coating of gutta-percha is desirable to facilitate bonding between the dentinal wall and root canal filling materials. Commercially available examples are such as the EndoReZTM system comprising resin-coated gutta-percha cones and resin sealer, or the Active GP PIusTM model comprising glass ionomer-coated gutta-percha cones and glass ionomer sealer.

Recent researches are addressing the use of bioactive material, hydroxyapatite (HA) as coating, especially for biological applications. As hydroxyapatite is the main inorganic constituent in bone and teeth, having the chemical composition of Ca₁₀(PO₄)₆(OH)₂, the use of HA and calcium phosphates in dental applications has evidently promote tissue growth and survival of cells, enhance fixation, biocompatibility, bioactivity, stability, and provide a more long-term result. In addition, the biomimetic approach permits the interaction between root dentin and the artificial structure, and enables the formation of a hermetic seal, which in turn becomes a barrier to reinfection by bacteria and prevents microleakage. Other available bone growth-promoting substances include: bioglass, calcium phosphate, Portland cement, tricalcium phosphate, a di- or polyphosphoric acid, an anti-estrogen, a sodium fluoride preparation, a substance having a phosphate to calcium ratio similar to natural bone, calcium hydroxide, and other suitable calcium-containing compounds. A bone growth-promoting substance may be in the form of a particulate or fiber filler in nano, micro, or macro form, or is composed of mixtures of bone chips, bone crystals, or mineral fractions of bone or teeth.

Among the various methods for application of coating, plasma spraying is the most conventional, involving high processing temperatures and a plasma created by argon gas, to spray molten materials onto a surface. Although plasma spraying is in current practice the most common method for deposition of hydroxyapatite as a coating film, this method is not suitable for application onto gutta-percha due to extremely high processing temperatures—gutta-percha has low melting point, and therefore low heat resistance. According to Cheang and Khor (1996), the problems also pertinent to plasma-sprayed hydroxyapatite coatings include the production of an amorphous phase (known as amorphous CaP) and other non-bioactive calcium phosphate phases. Other issues of plasma-spraying identified are such as the variation in bond strength between the coatings and metallic substrates, alterations in HA structure due to the coating process, and poor adhesion between the coatings and metallic substrates. In a review by Ong and Chan (1999), it has also been reported that plasma spraying of HA results in thick and brittle coatings, which are undesirable attributes. Other prior arts for generation of hydroxyapatite coating, as mentioned in EP Patent 0389713, include sputtering, sintering, sol-gel, and electrophoretic methods.

Reference may be made to Mutsuzaki et al (2013) on the plasma and precursor (amorphous calcium phosphate) mediated biomimetic process for forming apatite layer on Leeds-Keio artificial ligament, intended for ligament repair. In this study, the specimen with surface modified by plasma followed by precoating with amorphous calcium phosphate (ACP) was immersed in simulated body fluid (SBF). The resulting apatite-coated artificial ligament exhibited improved osseointegration and hence, is useful for ligament reconstructions. When immersed in SBF solution, the ACP and other metastable calcium phosphates are converted to apatite, which is the most stable crystalline phase in neutral solution.

U.S. Pat. No. 6,733,503 discloses a biomimetic method for coating medical implants, including dental prostheses, by immersing implants in highly concentrated aqueous solutions of magnesium, calcium, carbonate and phosphate ions at low temperature, through which a gaseous weak acid, such as carbon dioxide gas, is passed and the solution is degassed and the carbonated calcium phosphate coating is allowed to precipitate onto implant. The coating may be applied to any medical implant, inorganic, metallic, or organic in composition. The process involves the nucleation of carbonated calcium phosphate crystals on the surface of implantable devices; whereby a thin carbonated calcium phosphate layer can serve as seed crystals for the formation of subsequent layers. Hence, it is similarly reported here that coatings prepared via biomimetic method exhibit osseointegrative and osseoinductive properties (i.e. effective bone apposition).

Reference may also be made to U.S. Patent Publication 2011/0008460, which indicated the biomimetic approach to include ‘static’ and ‘dynamic’ methods for introducing nano-scale hydroxyapatite onto a matrix material, to produce composite materials for in vivo applications. A ‘static’ method refers to depositing pre-made HA particles on the matrix material, while a ‘dynamic’ method refers to the formation of HA on the matrix material by first depositing calcium ions (e.g. from calcium hydroxide) followed by a reaction with phosphate ions (e.g. from tribasic phosphate salt) to promote the mineralization of HA. Preferably, the solution for reaction as the calcium and phosphate ion source also contains one or more of water, buffer, solvent, simulated body fluid (SBF), or fortified cell medium, with or without serum. The exemplary matrix materials in preparing the composite include demineralized bone, mineralized bone, collagen, silks, polymeric materials, and combinations thereof. Therefore, according to the related literature and prior arts described, the biomimetic approach for coating of hydroxyapatite and calcium phosphates onto gutta-percha substrate is potentially able to circumvent the problems presented by conventional coating methods, and particularly is a promising candidate for dental applications.

### SUMMARY OF INVENTION

The object of the present invention is to produce a layer of coating comprising hydroxyapatite and tricalcium phosphate onto gutta-percha cones, through the biomimetic route, for use as root canal obturation matter. Hydroxyapatite (HA), Ca₁₀(PO₄)₆(OH)₂, is a calcium phosphate complex that is the primary mineral component of bone, found in a crystallized lattice-like form which provides rigidity. Hence, due to the biological functions of hydroxyapatite, the substance is commonly applied as composites, either by the introduction of HA nanoparticles within matrices or by the mineralization of HA on the surface of suitable substrates. To incorporate the beneficial characteristics of HA onto gutta-percha surface, formation of the coating occurs in simulated body fluid (SBF) in the present biomimetic technique.

The method proposed for coating briefly comprises the following steps: (i) pretreatment of gutta-percha cones with sodium hydroxide, (ii) vertical immersion of gutta-percha cones in SBF for duration of 10 days with replacement of solution every 48 hours, at physiological pH and temperature (i.e. incubation at 37° C. and pH level of 7.4), followed by (iii) nucleation process to form thin and uniform layer of micro-crystalline hydroxyapatite. The specific concentrations of ions present in the SBF solution are the following: 27 mM HCO₃⁻, 2.5 mM Ca²⁺, 1.0 mM HPO₄²⁻, 142 mM Na⁺, 125 mM Cl⁻, 5 mM K⁺, 1.5 mM Mg²⁺, and 0.5 mM SO₄²⁻. In this biomimetic method, nucleation occurs by control of pH and exposure time to coating solution. By changing the SBF solution every 48 hours within 10 days, the pH level of the reaction is maintained.

In the SBF solution, at physiological conditions, carbonated calcium phosphate aggregated on the surface of pretreated gutta-percha is metastable and will be converted into crystalline apatite. Once nuclei apatite are formed, they spontaneously grow by consuming the calcium and phosphate ions from the SBF solution, eventually forming a stable, dense, thin and uniform crystalline hydroxyapatite layer throughout the GP cones. The present inventors have demonstrated that the biomimetic method generated a strong adhesion between the bioactive coating and polymer substrate, measured in the critical load range of 431.61-1002.15 mN. Also, an in-vitro evaluation of sealing ability and bonding strength of the hydroxyapatite and tricalcium phosphate coated GP showed significant improvements in both characteristics when compared to uncoated GP and the resin-coated GP system, EndoReZ™. As the coating is formed though the biomimetic procedure, properties of interest such as biocompatibility and improved adhesion are deposited on the surface of the gutta-percha cones, to be further applied as filling matter for the restoration of the root canal system.

### DESCRIPTION OF THE EMBODIMENTS

The biomimetic method for coating of hydroxyapatite and tricalcium phosphate onto gutta-percha substrate is introduced in the present invention, in view of other current methods for coating such as plasma-spraying, sputtering, sintering, sol-gel, and electrophoretic methods. The application of hydroxyapatite as coating through the biomimetic route is prevalent in the medical industry, and is considered an attractive approach for bone and tissue engineering. Thus, in the present invention, said coating is developed by a biomimetic approach for the production of a root canal filling matter.

### EXAMPLE 1

In one embodiment of the present invention, the GP cones (ISO colour coded, Dentsply, Malliefier, USA) are pretreated with 5 M sodium hydroxide at temperature of 60° C., for 24 hours. Prior to the pretreatment process, the cones are abraded with #1000 SiC paper (FEPA P#1000, Struers, USA), and washed three times, with acetone, ethanol, and deionized water, respectively, in an ultrasonic bath (WiseClean, Korea). Following pretreatment, the cones are washed with deionized water and dried at 40° C. The pretreatment step is introduced to generate hydroxyl (OH) groups on the surface of GP cones. It is observed that the concentration of carboxyl and hydroxyl groups onpolymer surface most likely have a significant effect on speed and mechanism of calcium phosphate nucleation (Colovic et al, 2011). With high density of carboxyl and hydroxyl groups, there is a high density of nucleation sites. Hence the presence of OH groups is a factor which can influence the growth behavior of apatite crystals. In addition, Takeuchi et al (2005) reported that the arrangement of functional groups is important for the nucleation of hydroxyapatite in SBF, therefore the nucleation of HA on a substrate in a solution mimicking a body fluid is dependent on such structural functional group arrangements.

The concept of the biomimetic method is based on the finding that calcium phosphates are more soluble in mildly acidic medium than at neutral and basic pH, hence the precipitation of calcium phosphates occurs at neutral or basic pH; between solutions having the same concentrations of salts. An increase in pH value of solution can induce the following stages: under-saturation, super-saturation or the formation of metastable state, nucleation, and subsequent crystal growth. In the mechanism of heterogeneous nucleation, calcium phosphate nuclei can deposit onto a substrate when a solution has reached the super-saturation limit or the metastable condition. The predominant molecular form at the metastable phase is the intermediate product, amorphous calcium phosphate (ACP). It is proposed that the process of ACP formation in solution first involves the formation of Ca₉(PO₄)₆, also known as Posner's clusters, or CaP clusters, which then aggregates randomly to produce larger spherical particles or globules. These nano-clusters are found to be always present in simulated body fluid (SBF) solutions, and the insertion of suitable alkali-treated substrate surface into the solutions will stimulate the hexagonal packing of the nano-clusters to form apatitic CaP precipitates. At the metastable state, heterogeneous nucleation is favoured by the energy stabilization of nucleus on the substrate. Subsequently, the growth of the nucleated hydroxyapatite film will occur by simultaneous attraction of calcium and phosphate ions from the SBF solution. Finally, the high density of nucleation then ensures a uniform deposition of carbonated calcium phosphate crystals onto the surface of GP cones.

### EXAMPLE 2

In the present invention, the pretreated GP cones are immersed in SBF for duration of 10 days with replacement of the solution every 48 hours. The consumed ions of SBF are replaced with SBF having calcium and phosphate ions. SBF is a solution with inorganic ion concentrations almost equal to those in human blood plasma. The specific concentrations of ions present in the SBF solution prepared in this invention are the following: 27 mM HCO₃⁻, 2.5 mM Ca²⁺, 1.0 mM HPO₄²⁻, 142 mM Na⁺, 125 mM Cl⁻, 5 mM K⁺, 1.5 mM Mg²⁺, and 0.5 mM SO₄²⁻. This formulation has a Ca/P molar ratio of 2.5, and an ionic strength of 160.5 mM. Preferably, the physiological environment for formation of coating is kept at pH 7.4 and temperature of 37° C. The maintenance of ambient pH from the time of preparation through to the completion of the hydroxyapatite film formation is achieved by replacement of SBF solution in incubators, every 48 hours within 10 days. The buffering agent present in the SBF solution is tris-hydroxymethyl-aminomethane, with chemical formula (CH₂OH)₃CNH₂. The buffering agent TRIS is reported to form soluble complexes with several cations, including Ca²⁺, which results in the reduced concentration of free Ca²⁺ ions available for the real time calcium phosphate coating (Jalota et al, 2006). More importantly, the buffering action of TRIS and added hydrochloric acid (HCl) in SBF allows the maintenance of pH of the system over the range of pH 7.2 to pH 7.4. With respect to the function of TRIS in formation of coating, the buffer system has a proton buffering capacity in which HCO₃⁻ ions are incorporated into the structures of apatitic calcium phosphates in the form of CO₃²⁻ ions. In addition, the working pH of 7.4 is near the lower end of the buffering capacity of TRIS, and this facilitates the formation of carbonated apatitic CaP clusters in SBF solutions.

The biomimetic method applied on gutta-percha cones lead to the deposition of a micro-crystalline, bone-like apatite layer of 16 μm in thickness. The preferred range of thickness of the coating is between 14 to 19 μm; coating of this thickness may reduce the stress imposed on the coating and may enhance the bonding of coating to substrate. The present inventors have demonstrated that the biomimetic method generated a strong adhesion between the bioactive coating and gutta-percha substrate, measured in the critical load range of 431.61-1002.15 mN. A coating that has greater adhesive strength to the substrate is more difficult to delaminate, and results in higher critical load. Analyses using Fourier transform infrared spectroscopy (FTIR), X-ray Diffraction (XRD), and scanning electron microscope (SEM) are performed to characterize the chemical composition, morphology and structure of the coatings, and experimental data has confirmed that the coatings produced consist of carbonated calcium phosphates and hydroxyapatite.

The obturation technique implemented for the present invention is the matching single cone technique, and coated GP cones are obturated with the Endosequence BC sealer (Endosequence®, Brasseler, USA). An in vitro evaluation of sealing ability and bonding strength of the hydroxyapatite and tricalcium phosphate coated GP showed significant improvements in both characteristics when compared to uncoated GP and the resin-coated GP system, EndoReZ™. It is known that biomimetic processes often take place at ambient temperature and results in deposition of calcium phosphate that resembles one, or a combination of the numerous naturally occurring calcium phosphate compositions, therefore, mineralized hydroxyapatite on the gutta-percha cones is able to enhance bone-bonding effects and promote the growth of new bone and tissue surrounding the root canal. Improved sealing ability and bonding strength are significant in the formation of a hermetic seal. This is so that microleakage and adverse bacterial migration in the treated teeth can be prevented, which otherwise may be a cause for reinfection. In addition to the biological functions described above, heat treatment is not required for the biomimetic approach, and no intricate and expensive equipment are necessary. Hence, these aspects of the coated GP provide the solutions, leading to improved integrity as root canal filling matter. The present invention, involving a biomimetic method for coating of bioactive hydroxyapatite and tricalcium phosphate onto pre-treated gutta-percha substrate in SBF solution, is hence, a promising pathway in the fabrication of a root canal filling matter to be further applied in endodontic treatments and restorations.

### LIST OF NON-PATENT CITATIONS

Cheang, P. & Khor, K. A. (1996), ‘Addressing processing problems associated with plasma spraying of hydroxyapatite coatings’, Biomaterials, Vol. 17, No. 5, pp. 537-544.
Colovic, B., Markovic, D. & Jokanovic, V. (2011), ‘Nucleation of biomimetic hydroxyapatite’, Serbian Dental Journal, Vol. 58, No. 1, pp. 7-15.
Jalota, S., Bhaduri, S. B. & Tas, A. C. (2006), ‘Effect of carbonate content and buffer type on calcium phosphate formation in SBF solutions’, Journal of Materials Science: Materials in Medicine, Vol. 17, pp. 697-707.
Mutsuzaki, H., Yokoyama, Y., Ito, A. & Oyane, A. (2013), ‘Formation of apatite coatings on an artificial ligament using a plasma- and precursor-assisted biomimetic process’, International Journal of Molecular Sciences, Vol. 14, pp. 19155-19168.
Ong, J. L. & Chan, D. C. N. (1999), ‘Hydroxyapatite and their use as coatings in dental implants: a review’, Critical Reviews in Biomedical Engineering, Vol. 28, No. 5 & 6, pp. 1-41.
Takeuchi, A., Ohtsuki, C., Miyazaki, T., Kamitakahara, M., Ogata, S., Yamazaki, M., Furutani, Y., Kinoshita, H. & Tanihara, M. (2005), ‘Heterogeneous nucleation of hydroxyapatite on protein: structural effect of silk sericin’, Journal of the Royal Society Interface, Vol. 2, pp. 373-378.

1. A dental composite for root canal filling matter, comprising:
a gutta-percha substrate,
characterized in that,
a micro-crystalline hydroxyapatite and tricalcium phosphate coating is adhered onto the gutta-percha substrate.

2. The composite of claim 1, wherein the substrate is cone shaped.

3. The composite of claim 1, wherein the coating has a preferred thickness between 14 and 19 μm.

4. A method of preparing dental composite, comprising:
immersing gutta-percha cones in simulated body fluid, said fluid contains calcium and phosphate ions, for a predetermined period of time under physiological conditions; and
replacing the consumed ions of simulated body fluid with simulated body fluid having calcium and phosphate ions, at an interval of time.

5. The method of claim 4, wherein the immersing of gutta-percha cones in simulated body fluid is performed for duration of 10 days, at pH 7.4 and temperature 37° C., and replacing the simulated body fluid is performed every 48 hours within 10 days.

6. The method of claim 4, wherein the step of immersing the gutta-percha cones in simulated body fluid involves using simulated body fluid comprising: calcium, phosphate, carbonate, sodium, chloride, potassium, magnesium, and sulphate ions, with TRIS buffer and hydrochloric acid acting as buffering agents.

7. The method of claim 4, further comprising pretreatment step of: exposing the gutta-percha cones surface to sodium hydroxide, for the production of hydroxyl functional groups on the surface of gutta-percha cones, prior to immersing substrate in simulated body fluid.

8. The method of claim 7, wherein the pretreatment is performed at preferred concentration of sodium hydroxide is 5 M.

9. The method of claim 7, wherein the pretreatment is performed at preferred condition which include: immersing gutta-percha cones in sodium hydroxide for a period of 24 hours and at temperature of 60° C.
